(19) United States
(12) Patent Application Publication
(10) Pub. No.: US 2021/0398610 A1
(43) Pub. Date: Dec. 23, 2021

(54) SIGNIFICANCE MODELING OF CLONAL-LEVEL ABSENCE OF TARGET VARIANTS

(71) Applicant: GUARDANT HEALTH, INC., Redwood City, CA (US)

(72) Inventors: Aliaksandr ARTSIOMENKA, Mountain View, CA (US); Aaron Isaac HARDIN, San Francisco, CA (US); Stephen FAIRCLOUGH, Redwood City, CA (US); Marcin SIKORA, Redwood City, CA (US); Catalin BARBACIORU, Redwood City, CA (US)

(21) Appl. No.: 17/162,897

(22) Filed: Jan. 29, 2021

Related U.S. Application Data
(60) Provisional application No. 62/968,507, filed on Jan. 31, 2020.

(51) Int. Cl. G16B 20/20; G16H 10/40; G16H 50/70; G16H 20/10

(52) U.S. Cl. CPC G16B 20/20; G16H 20/10; G16H 50/70; G16H 10/40

(57) ABSTRACT

Provided herein are methods of making negative predictions. In some aspects, methods of determining that a first target nucleic acid variant is absent at a first genetic locus in a cell-free nucleic acid (cfNA) sample obtained from a subject having a given cancer type at least partially using a computer are provided. Certain of these methods include determining that the first target nucleic acid variant is not detected in the cfNA sample obtained from the subject, generating, by the computer, at least one tumor fraction based value; generating, by the computer, at least one mutual exclusivity value; and determining that the first target nucleic acid variant is absent at the first genetic locus in the cfNA sample using the tumor fraction based value and/or the mutual exclusivity value. Additional methods and related systems and computer readable media are also provided.

### CROSS-REFERENCE

This application claims the benefit of the priority date of U.S. Provisional Patent Application No. 62/968,507, filed on Jan. 31, 2020, which is incorporated by reference in its entirety for all purposes.

### BACKGROUND

In advanced colorectal cancer (CRC), guidelines recommend the use of anti-EGFR therapies only in patients whose tumors are wild-type for KRAS, NRAS, and BRAF. To date, cell-free circulating tumor DNA (ctDNA) tests have been used as rule-in tests for positive detection of tumor-derived genomic alterations and microsatellite instability (MSI) with high concordance to tissue sequencing (Gupta et al., Oncologist, 24:1-9 (2019), Parikh et at., Nat Med., 25(9):1415-1421 (2019)). However, the ability to rule out such mutations has been limited due to the potential of low ctDNA shedding impacting sensitivity of detection. Using ctDNA or other nucleic acids to determine the wild-type status of specific genes within a tumor with high confidence would facilitate timely therapeutic decision making and avoid tissue biopsy for confirmation of wild-type status.

Accordingly, there remains a need to identify genetic variants, or the absence thereof, to diagnose and/or guide the treatment of diseases that are detectable through genetic analysis, especially from cell-free nucleic acid (cfNA) samples.

### SUMMARY

The disclosure relates to technology that generates a precision diagnosis based on a determination of various states of nucleic acids such as a DNA or RNA from a genome, chromosome, or other genetic portion sequenced from a sample. Detection of a target variant may be instrumental in guiding treatment plans.

When a genetic variant is not detected, it may be equally important to determine whether the genetic variant was not detected because the variant is not actually present at the clonal level in a sample (a true negative result) or the genetic variant is actually present at the clonal level but was not detected (a false negative result). Described herein are improvements relating to significance modeling of negative predictions, such as whether a genetic variant was not detected or is not actually present in a sample. In particular examples, the significance modeling may generate and use computational estimates of tumor fraction (TF) of a tumor variant or mutation based on nucleic acid sequence reads generated from the sample.

Alternatively, or additionally, the significance modeling may determine and use the prevalence and/or diversity of other variants that are detected—or not detected—in the sample. For example, the significance modeling may use detection of covariance variants that co-occur with the target variant or mutually exclusive variants that usually do not co-occur with the target variant. A negative predictive value (“NPV”) may be generated based on the TF estimates and/or diversity of variants that are detected, or not detected, in the sample. The result may be used to provide a level of confidence in a negative diagnosis (e.g., an absence of a given variant at a locus of interest) and/or to further guide treatment plans based on the negative diagnosis. In the context of cancer diagnosis, for example, co-occurrence variants may include driver variants that tend to promote oncogenesis and mutually exclusive variants may include tumor suppressor variants that tend to suppress oncogenesis.

In one aspect, the present disclosure provides a method of determining a probability that a first variant of interest at a first locus is absent at a clonal level in a nucleic acid sample obtained from a subject. The method includes accessing a plurality of sequence reads of nucleic acids in the sample; and determining that the first variant has not been detected at the first locus in the sample based on the plurality of sequence reads. The method also includes generating a first likelihood value based on a probability that the first variant is absent at the clonal level and a second likelihood value based on a probability that the first variant is not absent at the clonal level; determining a quantitative value based on the first likelihood value and the second likelihood value; comparing the quantitative value to a threshold; and determining that the first variant of interest at the first locus is absent at the clonal level based on the comparison.

In one aspect, the present disclosure provides a method of determining that a first variant of interest at a first locus is absent at a clonal level in a cell-free nucleic acid (cfNA) sample of a human subject (and negative predictions). The method includes accessing a plurality of sequence reads of the cfNA sample; and determining that the first variant has not been detected at the first locus in the sample based on the plurality of sequence reads. The method also includes generating a first likelihood value based on a probability that the first variant is absent at the clonal level and/or a second likelihood value based on a probability that the first variant is not absent at the clonal level; and classifying that the first variant of interest at the first locus is absent at the clonal level based on the comparison.

In one aspect, the present disclosure provides a method of determining that a first variant of interest at a first locus is absent at a clonal level in a cell-free deoxyribonucleic acid (cfDNA) sample of a human subject (and negative predictions). The method includes accessing a plurality of sequence reads of the cfDNA sample; and determining that the first variant has not been detected at the first locus in the sample based on the plurality of sequence reads. The method also includes generating a first likelihood value based on a probability that the first variant is absent at the clonal level and/or a second likelihood value based on a probability that the first variant is not absent at the clonal level; determining a optionally, quantitative value based on the first likelihood value and/or the second likelihood value; comparing the quantitative value and/or the first likelihood value and/or the second likelihood value to a threshold; and determining (e.g., classifying or calling in this context) that the first variant of interest at the first locus is absent at the clonal level based on the comparison.

In some embodiments, generating the first likelihood value and the second likelihood value comprises: determining a tumor fraction estimate of the sample, wherein the first likelihood value and the second likelihood value is based on the tumor fraction estimate. In certain embodiments, determining the tumor fraction estimate comprises: determining a maximum mutant allele frequency (MAX MAF) of a tumor mutation in the sample. In some of these embodiments, determining the MAX MAF comprises determining a molecule count associated with the tumor mutation based on the plurality of sequence reads. In certain embodiments, generating the first likelihood value and the second likelihood value comprises: determining an allele frequency of at least a second variant, wherein the first likelihood value and the second likelihood value are based further on the allele frequency and the MAX MAF. In certain of these embodiments, the method further includes comparing the allele frequency with a second threshold that is based on the MAX MAF, wherein determining that the first variant of interest at the first locus is absent at the clonal level is based further on the comparison of the MAF with the second threshold. In certain of these embodiments, determining the allele frequency comprises: determining a first molecule count associated with the first variant based on the plurality of sequence reads. In some embodiments, determining the quantitative value comprises: accessing covariable information indicating a historical prevalence of one or more variants exhibiting co-occurrence and/or mutual exclusivity with the first variant, wherein the quantitative value is based on the covariable information. In some of these embodiments, the method further includes determining a prevalence of at least a second variant in the cfDNA sample, wherein the quantitative value is based further on the covariable information.

In certain embodiments, determining the quantitative value comprises: accessing covariable information indicating a historical prevalence of one or more variants exhibiting co-occurrence and/or mutual exclusivity with the first variant, wherein the quantitative value is based on the covariable information. In some of these embodiments, the method further includes determining a prevalence of at least a second variant in the cfDNA sample, wherein the quantitative value is based further on the prevalence of the second variant. In certain embodiments, the quantitative value is based on the ratio of the first likelihood value to the second likelihood value. In certain embodiments, the method further comprises determining a level of confidence that the first variant is absent at the clonal level in the cfDNA sample based on the quantitative value. In some embodiments, the method further comprises determining generating a treatment plan to treat a disease in the human subject. In some of these embodiments, the disease is cancer. In certain embodiments, the method further comprises determining a prevalence of at least a second variant in the cfDNA sample; and adjusting the quantitative value based on the prevalence of at least a second variant in the cfDNA sample.

In another aspect, the present disclosure provides a method of determining that a first target nucleic acid variant is absent at a first genetic locus in a cell-free nucleic acid (cfNA) sample obtained from a subject having a given cancer type at least partially using a computer. The method comprises determining that the first target nucleic acid variant at the first genetic locus is not detected in the cfNA sample; determining, by the computer, a coverage of the first genetic locus from sequence information generated from the cfNA sample; and determining, by the computer, a tumor fraction from the sequence information generated from the cfNA sample. The method also includes determining, by the computer, a probability that the first target nucleic acid variant is not absent at the first genetic locus in the cfNA sample from the coverage and the tumor fraction to generate a quantitative value; and determining (e.g., classifying or calling in this context) that the first target nucleic acid variant is absent at the first genetic locus in the cfNA sample when the quantitative value differs from a threshold value.

In another aspect, the present disclosure provides a method of determining that a first target nucleic acid variant is absent at a first genetic locus in a cell-free nucleic acid (cfNA) sample obtained from a subject at least partially using a computer. The method comprises: determining that the first target nucleic acid variant is not detected in the cfNA sample obtained from the subject to generate a first test result; determining that at least a second target nucleic acid variant is detected in the cfNA sample obtained from the subject to generate a second test result; and determining, by the computer, a first probability that the first target nucleic acid variant is absent in the cfNA sample given the second test result and/or a second probability that the first target nucleic acid is not absent in the cfNA sample given the second test result. The method also includes generating, by the computer, a quantitative value using the first probability, the second probability, and/or a ratio thereof, and determining (e.g., classifying or calling in this context) that the first target nucleic acid variant is absent at the first genetic locus in the cfNA sample when the quantitative value differs from a threshold value.

In another aspect, the present disclosure provides a method of determining that a first target nucleic acid variant is absent at a first genetic locus in a cell-free nucleic acid (cfNA) sample obtained from a subject having a given cancer type at least partially using a computer. The method comprises: determining that the first target nucleic acid variant is not detected in the cfNA sample obtained from the subject; generating, by the computer, at least one tumor fraction based value; generating, by the computer, at least one mutual exclusivity value; and determining (e.g., classifying or calling in this context) that the first target nucleic acid variant is absent at the first genetic locus in the cfNA sample using the tumor fraction based value and/or the mutual exclusivity value.

In some embodiments, the quantitative value is less than the threshold value, whereas in other embodiments, the quantitative value is greater than the threshold value. In certain embodiments, the quantitative value comprises a log likelihood ratio (LLR) threshold value. Typically, the first and second test results are dependent upon one another. In certain embodiments, the methods disclosed herein include determining that a plurality of other selected target nucleic variants are absent at one or more other genetic loci (e.g., a panel of selected or target loci).

In certain embodiments, the methods include determining that the first target nucleic acid variant is absent at the first genetic locus in a plurality of reference cfNA samples to generate the threshold value. In some of these embodiments, the threshold value comprises a clonality or a sub-clonality threshold value. In some embodiments of the methods disclosed herein, the first target nucleic acid variant comprises a driver mutation. In certain embodiments, the methods further include administering one or more therapies to the subject based upon the determination that the first target nucleic acid variant is absent at the first genetic locus in the cfNA sample. In some embodiments, the methods include estimating a probability of detecting the first target nucleic acid variant at the first genetic locus in the cfNA sample using the tumor fraction and a binomial model. In certain of these embodiments, the binomial model comprises information about the given cancer type and/or the second target nucleic acid variant. Other models are also optionally used.

In some embodiments of the methods disclosed herein, the determination that the first target nucleic acid variant is absent at the first genetic locus in the cfNA sample indicates that the first genetic locus is wild type. In certain embodiments, the given cancer type is colorectal cancer, wherein the first genetic locus is KRAS, BRAF, or NRAS, and wherein the determination that the first target nucleic acid variant is absent at the first genetic locus in the cfNA sample indicates that the first genetic locus is wild type KRAS, BRAF, or NRAS. In certain of these embodiments, the methods further include administering Cetuximab and/or Panitumumab to the subject. In some embodiments, the cfNA comprises cfDNA and/or cfRNA.

In certain embodiments, the methods disclosed herein further include repeating the method one or more times to monitor whether the first target nucleic acid variant is absent at the first genetic locus in different cfNA samples obtained from the subject at different time points. In certain embodiments, the methods further comprise performing one or more additional tests to confirm or refute the determination that the first target nucleic acid variant is absent at the first genetic locus in the cfNA sample. In some embodiments, the methods include determining a maximum mutant allele frequency (MAX MAF) for the cfNA sample and using the MAX MAF as an estimate of the tumor fraction. In certain embodiments, the methods include determining that first target nucleic acid variant at the first genetic locus is not detected in the cfNA sample based upon a plurality of sequencing reads obtained from the cfNA sample. In some embodiments, the methods comprise determining that the first target nucleic acid variant is absent at a clonal level in the cfNA sample. In certain embodiments, the methods include generating a first likelihood value based on the first probability and a second likelihood value based on the second probability. In certain embodiments, the methods include determining the quantitative value based on the first likelihood value and the second likelihood value.

In some embodiments of the methods disclosed herein, generating the first likelihood value and the second likelihood value comprises determining the tumor fraction estimate of the cfNA sample, wherein the first likelihood value and the second likelihood value is based on the tumor fraction estimate. In certain embodiments, the methods include determining the tumor fraction estimate comprises determining a maximum mutant allele frequency (MAX MAF) of a tumor mutation in the cfNA sample. In certain embodiments, the methods include determining the MAX MAF comprises determining a molecule count associated with the tumor mutation based on the plurality of sequence reads. In some embodiments, the methods include generating the first likelihood value and the second likelihood value comprises determining an allele frequency of at least a second variant, wherein the first likelihood value and the second likelihood value are based further on the allele frequency and the MAX MAF. In some of these embodiments, the methods further comprise comparing the allele frequency with a second threshold that is based on the MAX MAF, wherein determining that the first target nucleic acid variant of interest at the first genetic locus is absent at the clonal level is based further on the comparison of the MAF with the second threshold.

In some embodiments, determining the first allele frequency comprises determining a first molecule count associated with the first target nucleic acid variant based on the plurality of sequence reads. In certain embodiments, determining the quantitative value comprises accessing covariable information indicating a historical prevalence of one or more variants exhibiting co-occurrence and/or mutual exclusivity with the first variant, wherein the quantitative value is based on the covariable information. In some embodiments, the methods further comprise determining a prevalence of at least the second target nucleic acid variant in the cfDNA sample, wherein the quantitative value is based further on the covariable information. In certain embodiments, the methods include determining the quantitative value comprises accessing covariable information indicating a historical prevalence of one or more variants exhibiting co-occurrence and/or mutual exclusivity with the first target nucleic acid variant, wherein the quantitative value is based on the covariable information. In some embodiments, the methods further comprise determining a prevalence of at least the second target nucleic acid variant in the cfNA sample, wherein the quantitative value is based further on the prevalence of the second target nucleic acid variant. In some of these embodiments, the quantitative value is based on the ratio of the first likelihood value to the second likelihood value. In certain of these embodiments, the methods further comprise determining a level of confidence that the first target nucleic acid variant is absent at a clonal level in the cfNA sample based on the quantitative value. In some of these embodiments, the methods further comprise determining a prevalence of at least the second target nucleic acid variant in the cfNA sample; and adjusting the quantitative value based on the prevalence of at least the second target nucleic acid variant in the cfNA sample.

In some embodiments of the methods disclosed herein, the ratio comprises a log posterior probability ratio (LPPR) equal to a sum of a log likelihood tumor fraction value, a log likelihood mutual exclusivity value, and a log prior value. In certain embodiments, the first genetic locus or a second genetic locus comprises the second target nucleic acid variant. In certain embodiments, the quantitative value comprises a negative predictive value (NPV) score. In some embodiments, the given cancer type comprises lung cancer and the first target nucleic acid variant is a mutation in a gene selected from the group consisting of: EGFR, BRAF (e.g., V600E), ALK (e.g., fusions), ROS1 (e.g., fusions), and MET. In some embodiments, the given cancer type comprises colorectal cancer and the first target nucleic acid variant is a mutation in a gene selected from the group consisting of: KRAS (e.g., G12X, G13X, Q61X, K117N, A146P/146T/146V), BRAF, and NRAS.

In another aspect, the present disclosure provides a system comprising a controller comprising, or capable of accessing, computer readable media comprising non-transitory computer executable instructions which, when executed by at least one electronic processor, perform at least: accessing a plurality of sequence reads of the cfDNA sample; determining that the first variant has not been detected at the first locus in the sample based on the plurality of sequence reads; generating a first likelihood value based on a probability that the first variant is absent at the clonal level and a second likelihood value based on a probability that the first variant is not absent at the clonal level; determining a quantitative value based on the first likelihood value and the second likelihood value; comparing the quantitative value to a threshold; and determining (e.g., classifying or calling in this context) that the first variant of interest at the first locus is absent at the clonal level based on the comparison.

In another aspect, the present disclosure provides a system, comprising a controller comprising, or capable of accessing, computer readable media comprising non-transitory computer executable instructions which, when executed by at least one electronic processor, perform at least: accessing sequence information generated from a cell-free nucleic acid (cfNA) sample obtained from a subject having a given cancer type; determining that a first target nucleic acid variant at a first genetic locus is not detected in cfNA sample from the sequence information; determining a coverage of the first genetic locus from the sequence information; determining a tumor fraction from the sequence information; determining a probability that the first target nucleic acid variant is not absent at the first genetic locus in the cfNA sample from the coverage and the tumor fraction to generate a quantitative value; and determining (e.g., classifying or calling in this context) that the first target nucleic acid variant is absent at the first genetic locus in the cfNA sample when the quantitative value differs from a threshold value.

In another aspect, the present disclosure provides a system, comprising a controller comprising, or capable of accessing, computer readable media comprising non-transitory computer executable instructions which, when executed by at least one electronic processor, perform at least: accessing sequence information generated from a cell-free nucleic acid (cfNA) sample obtained from a subject; determining that the first target nucleic acid variant is not detected in the cfNA sample from the sequence information to generate a first test result; determining that at least a second target nucleic acid variant is detected in the cfNA sample from the sequence information to generate a second test result; determining a first probability that the first target nucleic acid variant is absent in the cfNA sample given the second test result and/or a second probability that the first target nucleic acid is not absent in the cfNA sample given the second test result; generating a quantitative value using the first probability, the second probability, and/or a ratio thereof, and determining (e.g., classifying or calling in this context) that the first target nucleic acid variant is absent at the first genetic locus in the cfNA sample when the quantitative value differs from a threshold value.

In another aspect, the present disclosure provides a system, comprising a controller comprising, or capable of accessing, computer readable media comprising non-transitory computer executable instructions which, when executed by at least one electronic processor, perform at least: accessing sequence information generated from a cell-free nucleic acid (cfNA) sample obtained from a subject; determining that the first target nucleic acid variant is not detected in the cfNA sample from the sequence information; generating at least one tumor fraction based value; generating at least one mutual exclusivity value; and determining (e.g., classifying or calling in this context) that the first target nucleic acid variant is absent at the first genetic locus in the cfNA sample using the tumor fraction based value and/or the mutual exclusivity value.

In another aspect, the present disclosure provides a computer readable media comprising non-transitory computer executable instruction which, when executed by at least electronic processor perform at least: accessing a plurality of sequence reads of the cfDNA sample; determining that the first variant has not been detected at the first locus in the sample based on the plurality of sequence reads; generating a first likelihood value based on a probability that the first variant is absent at the clonal level and a second likelihood value based on a probability that the first variant is not absent at the clonal level; determining a quantitative value based on the first likelihood value and the second likelihood value; comparing the quantitative value to a threshold; and determining (e.g., classifying or calling in this context) that the first variant of interest at the first locus is absent at the clonal level based on the comparison.

In another aspect, the present disclosure provides a computer readable media comprising non-transitory computer executable instruction which, when executed by at least electronic processor perform at least: accessing sequence information generated from a cell-free nucleic acid (cfNA) sample obtained from a subject having a given cancer type; determining that a first target nucleic acid variant at a first genetic locus is not detected in cfNA sample from the sequence information; determining a coverage of the first genetic locus from the sequence information; determining a tumor fraction from the sequence information; determining a probability that the first target nucleic acid variant is not absent at the first genetic locus in the cfNA sample from the coverage and the tumor fraction to generate a quantitative value; and determining (e.g., classifying or calling in this context) that the first target nucleic acid variant is absent at the first genetic locus in the cfNA sample when the quantitative value differs from a threshold value.

In another aspect, the present disclosure provides a computer readable media comprising non-transitory computer executable instruction which, when executed by at least electronic processor perform at least: accessing sequence information generated from a cell-free nucleic acid (cfNA) sample obtained from a subject; determining that the first target nucleic acid variant is not detected in the cfNA sample from the sequence information to generate a first test result; determining that at least a second target nucleic acid variant is detected in the cfNA sample from the sequence information to generate a second test result; determining a first probability that the first target nucleic acid variant is absent in the cfNA sample given the second test result and/or a second probability that the first target nucleic acid is not absent in the cfNA sample given the second test result; generating a quantitative value using the first probability, the second probability, and/or a ratio thereof, and determining (e.g., classifying or calling in this context) that the first target nucleic acid variant is absent at the first genetic locus in the cfNA sample when the quantitative value differs from a threshold value.

In another aspect, the present disclosure provides a computer readable media comprising non-transitory computer executable instruction which, when executed by at least electronic processor perform at least: accessing sequence information generated from a cell-free nucleic acid (cfNA) sample obtained from a subject; determining that the first target nucleic acid variant is not detected in the cfNA sample from the sequence information; generating at least one tumor fraction based value; generating at least one mutual exclusivity value; and determining (e.g., classifying or calling in this context) that the first target nucleic acid variant is absent at the first genetic locus in the cfNA sample using the tumor fraction based value and/or the mutual exclusivity value.

In some embodiments of the system or computer readable media disclosed herein, the quantitative value is less than the threshold value, whereas in other exemplary embodiments, the quantitative value is greater than the threshold value. In some of these embodiments, the first and second test results are dependent upon one another. In certain of these embodiments, the non-transitory computer executable instructions include determining that a plurality of other selected target nucleic variants are absent at one or more other genetic loci. In some of these embodiments, the quantitative value comprises a log likelihood ratio (LLR) threshold value. In certain of these embodiments, the non-transitory computer executable instructions include determining that the first target nucleic acid variant is absent at the first genetic locus in a plurality of reference cfNA samples to generate the threshold value. In some of these embodiments, the threshold value comprises a clonality or sub-clonality threshold value. In some of these embodiments, the first target nucleic acid variant comprises a driver mutation. In some of these embodiments, the instructions further perform at least: outputting one or more therapy recommendations for the subject based upon the determination that the first target nucleic acid variant is absent at the first genetic locus in the cfNA sample.

In some embodiments of the system or computer readable media disclosed herein, the instructions further perform at least: estimating a probability of detecting the first target nucleic acid variant at the first genetic locus in the cfNA sample using the tumor fraction and a binomial model. In some of these embodiments, the instructions further perform at least: determining a maximum mutant allele frequency (MAX MAF) for the cfNA sample and using the MAX MAF as an estimate of the tumor fraction. In some of these embodiments, wherein the instructions further perform at least: determining that the first target nucleic acid variant is absent at a clonal level in the cfNA sample. In certain of these embodiments, the instructions further perform at least: generating a first likelihood value based on the first probability and a second likelihood value based on the second probability. In certain of these embodiments, the instructions further perform at least: determining the quantitative value based on the first likelihood value and the second likelihood value.

In some embodiments of the system or computer readable media disclosed herein, the instructions further perform at least: generating the first likelihood value and the second likelihood value by determining the tumor fraction estimate of the cfNA sample, wherein the first likelihood value and the second likelihood value is based on the tumor fraction estimate. In certain of these embodiments, the instructions further perform at least: determining the tumor fraction estimate by determining a maximum mutant allele frequency (MAX MAF) of a tumor mutation in the cfNA sample. In certain of these embodiments, the instructions further perform at least: determining the MAX MAF by determining a molecule count associated with the tumor mutation based on the plurality of sequence reads. In certain of these embodiments, the instructions further perform at least: generating the first likelihood value and the second likelihood value by determining an allele frequency of at least a second variant, wherein the first likelihood value and the second likelihood value are based further on the allele frequency and the MAX MAF. In some of these embodiments, the instructions further perform at least: comparing the allele frequency with a second threshold that is based on the MAX MAF and determining that the first target nucleic acid variant of interest at the first genetic locus is absent at the clonal level based further on the comparison of the MAF with the second threshold. In some of these embodiments, the instructions further perform at least: determining the allele frequency by determining a first molecule count associated with the first target nucleic acid variant based on the plurality of sequence reads.

In some embodiments of the system or computer readable media disclosed herein, the instructions further perform at least: determining the quantitative value by accessing covariable information indicating a historical prevalence of one or more variants exhibiting co-occurrence and/or mutual exclusivity with the first variant, wherein the quantitative value is based on the covariable information. In some of these embodiments, the instructions further perform at least: determining a prevalence of at least the second target nucleic acid variant in the cfDNA sample, wherein the quantitative value is based further on the covariable information. In some of these embodiments, the instructions further perform at least: determining the quantitative value by accessing covariable information indicating a historical prevalence of one or more variants exhibiting co-occurrence and/or mutual exclusivity with the first target nucleic acid variant, wherein the quantitative value is based on the covariable information. In certain of these embodiments, the instructions further perform at least: determining a prevalence of at least the second target nucleic acid variant in the cfNA sample, wherein the quantitative value is based further on the prevalence of the second target nucleic acid variant. In certain of these embodiments, the instructions further perform at least: determining a level of confidence that the first target nucleic acid variant is absent at a clonal level in the cfNA sample based on the quantitative value. In certain of these embodiments, the instructions further perform at least: determining a prevalence of at least the second target nucleic acid variant in the cfNA sample; and adjusting the quantitative value based on the prevalence of at least the second target nucleic acid variant in the cfNA sample. In certain of these embodiments, the ratio comprises a log posterior probability ratio (LPPR) equal to a sum of a log likelihood tumor fraction value, a log likelihood mutual exclusivity value, and a log prior value.

In some embodiments, the results of the systems and methods disclosed herein are used as an input to generate a report. The report may be in a paper or electronic format. For example the classification that a first variant of interest at a first locus is absent at a clonal level, as obtained by the methods and systems disclosed herein, can be displayed directly in such a report. Alternatively or additionally, diagnostic information or therapeutic recommendations based on the probability that a first variant of interest at a first locus is absent at a clonal level can be included in the report.

Where a determination is based on a quantitative value differing from a threshold value, the quantitative value used in this determination may be less than the threshold value or greater than the threshold value, depending on the nature of the threshold value. Thus the quantitative value either meets the threshold or does not.

In certain aspects, the present disclosure provides for a method of treating a disease in the subject, the method comprising: accessing a plurality of sequence reads of a cell-free deoxyribonucleic acid (cfDNA) sample obtained from the subject; determining that a first variant of interest at a first locus has not been detected at the first locus in the cfDNA sample based on the plurality of sequence reads; generating a first likelihood value based on a probability that the first variant is absent at a clonal level and/or a second likelihood value based on a probability that the first variant is not absent at the clonal level; determining a quantitative value based on the first likelihood value and/or the second likelihood value; comparing the quantitative value and/or the first likelihood value and/or the second likelihood value to a threshold; determining that the first variant of interest at the first locus is absent at the clonal level based on the comparison; and, administering one or more therapies to the subject based at least in part upon determining that the first variant of interest at the first locus is absent at the clonal level, thereby treating the disease in the subject. In certain embodiments, one or more therapies are discontinued being administered to the subject based at least in part upon determining that the first variant of interest at the first locus is absent at the clonal level, thereby treating the disease in the subject. In certain embodiments, the method described herein are performed on a plurality of subjects. In certain embodiments, a subset of the subjects are administered one or more therapies based at least in part upon determining that the first variant of interest at the first locus is absent at the clonal level, and another subset of the subjects are discontinued from one or more therapies that were previously administered to those subjects. In certain embodiments, a subject is administered a different therapy than a therapy that was previously administered to the subject based at least in part upon determining that the first variant of interest at the first locus is absent at the clonal level.

In certain aspects, the present disclosure provides for a method of treating a disease in a subject, the method comprising administering, or discontinuing administering, one or more therapies to the subject based at least in part upon a determination that a first variant of interest at a first locus is absent at a clonal level in a cell-free deoxyribonucleic acid (cfDNA) sample obtained from the subject, wherein the determination is produced by: accessing a plurality of sequence reads of the cfDNA sample; determining that the first variant has not been detected at the first locus in the sample based on the plurality of sequence reads; generating a first likelihood value based on a probability that the first variant is absent at the clonal level and/or a second likelihood value based on a probability that the first variant is not absent at the clonal level; determining a quantitative value based on the first likelihood value and/or the second likelihood value; comparing the quantitative value and/or the first likelihood value and/or the second likelihood value to a threshold; and determining that the first variant of interest at the first locus is absent at the clonal level based on the comparison.

In certain aspects, the present disclosure provides for a method of treating cancer in the subject, the method comprising: determining that the first target nucleic acid variant at the first genetic locus is not detected in cell-free nucleic acid (cfNA) sample obtained from the subject having the cancer; determining a coverage of the first genetic locus from sequence information generated from the cfNA sample; determining a tumor fraction from the sequence information generated from the cfNA sample; determining a probability that the first target nucleic acid variant is not absent at the first genetic locus in the cfNA sample from the coverage and the tumor fraction to generate a quantitative value; determining that the first target nucleic acid variant is absent at the first genetic locus in the cfNA sample when the quantitative value differs from a threshold value; and, administering, or discontinuing administering, one or more therapies to the subject based at least in part upon determining that the first target nucleic acid variant is absent at the first genetic locus in the cfNA sample, thereby treating the cancer in the subject.

In certain aspects, the present disclosure provides for a method of treating a cancer in a subject, the method comprising administering, or discontinuing administering, one or more therapies to the subject based at least in part upon a determination that a first target nucleic acid variant is absent at the first genetic locus in a cell-free deoxyribonucleic acid (cfDNA) sample obtained from the subject having the cancer, wherein the determination is produced by: determining that the first target nucleic acid variant at the first genetic locus is not detected in the cfNA sample; determining a coverage of the first genetic locus from sequence information generated from the cfNA sample; determining a tumor fraction from the sequence information generated from the cfNA sample; determining a probability that the first target nucleic acid variant is not absent at the first genetic locus in the cfNA sample from the coverage and the tumor fraction to generate a quantitative value; and, determining that the first target nucleic acid variant is absent at the first genetic locus in the cfNA sample when the quantitative value differs from a threshold value.

In certain aspects, the present disclosure provides a method of treating a disease in the subject, the method comprising: determining that a first target nucleic acid variant is not detected in a cell-free nucleic acid (cfNA) sample obtained from the subject to generate a first test result; determining that at least a second target nucleic acid variant is detected in the cfNA sample obtained from the subject to generate a second test result; determining a first probability that the first target nucleic acid variant is absent in the cfNA sample given the second test result and/or a second probability that the first target nucleic acid is not absent in the cfNA sample given the second test result; generating a quantitative value using the first probability, the second probability, and/or a ratio thereof; determining that the first target nucleic acid variant is absent at the first genetic locus in the cfNA sample when the quantitative value differs from a threshold value; and, administering, or discontinuing administering, one or more therapies to the subject based at least in part upon determining that the first target nucleic acid variant is absent at the first genetic locus, thereby treating the disease in the subject.

In certain aspects, the present disclosure provides for a method of treating a disease in a subject, the method comprising administering, or discontinuing administering, one or more therapies to the subject based at least in part upon a determination that a first target nucleic acid variant is absent at a first genetic locus in a cell-free nucleic acid (cfNA) sample obtained from a subject, wherein the determination is produced by: determining that the first target nucleic acid variant is not detected in the cfNA sample obtained from the subject to generate a first test result; determining that at least a second target nucleic acid variant is detected in the cfNA sample obtained from the subject to generate a second test result; determining a first probability that the first target nucleic acid variant is absent in the cfNA sample given the second test result and/or a second probability that the first target nucleic acid is not absent in the cfNA sample given the second test result; generating a quantitative value using the first probability, the second probability, and/or a ratio thereof; and, determining that the first target nucleic acid variant is absent at the first genetic locus in the cfNA sample when the quantitative value differs from a threshold value.

In certain aspects, the present disclosure provides for a method of treating cancer in the subject, the method comprising: determining that a first target nucleic acid variant is absent at a first genetic locus in a cell-free nucleic acid (cfNA) sample obtained from a subject having a given cancer type; generating at least one tumor fraction based value; generating at least one mutual exclusivity value; determining that the first target nucleic acid variant is absent at the first genetic locus in the cfNA sample using the tumor fraction based value and/or the mutual exclusivity value; and, administering, or discontinuing administering, one or more therapies to the subject based at least in part upon determining that the first target nucleic acid variant is absent at the first genetic locus in the cfNA sample, thereby treating the cancer in the subject.

In certain aspects, the present disclosure provides for a method of treating a cancer in a subject, the method comprising administering, or discontinuing administering, one or more therapies to the subject based at least in part upon a determination that a first target nucleic acid variant is absent at a first genetic locus in a cell-free nucleic acid (cfNA) sample obtained from a subject having a given cancer type, wherein the determination is produced by: determining that the first target nucleic acid variant is not detected in the cfNA sample obtained from the subject; generating at least one tumor fraction based value; generating at least one mutual exclusivity value; and, determining that the first target nucleic acid variant is absent at the first genetic locus in the cfNA sample using the tumor fraction based value and/or the mutual exclusivity value.

The various steps of the methods disclosed herein, or steps carried out by the systems disclosed herein, may be carried out at the same or different times, in the same or different geographical locations, e.g. countries, and/or by the same or different people.

### BRIEF DESCRIPTION OF THE FIGURES

FIG. 1 illustrates an example of a system for generating negative predictions of a target variant in a sample of a subject, according to an embodiment of the disclosure.

FIG. 2 illustrates a schematic diagram of inputs and outputs of a negative prediction analyzer, according to an embodiment.

FIG. 3 illustrates an example of a method for generating negative predictions of a target variant in a sample of a subject, according to an embodiment of the disclosure.

FIG. 4A illustrates a graph of a test hypothesis in which a target variant (the target variant) is absent (or present at sub-clonal MAF) from the sample, according to an embodiment.

FIG. 4B illustrates a graph of a null hypothesis in which the target variant is not absent in the sample, according to an embodiment.

### DEFINITIONS

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. Adapters of the same or different sequence can be linked to the respective ends of a nucleic acid molecule. In certain embodiments, an adapter of the same sequence is linked to the respective ends of the nucleic acid molecule except that the nucleic acid tag differs in its sequence. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Allele: As used herein, “allele” or “allelic variant” refers to a specific genetic variant at defined genomic location or locus. An allelic variant is usually presented at a frequency of 50% (0.5) or 100%, depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule having a sequence that can serve as a molecular identifier. For example, individual “barcode” sequences are typically added to each DNA fragment during next-generation sequencing (NGS) library preparation so that each read can be identified and sorted before the final data analysis.

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, KRAS, BRAF, NRAS, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Clonal: As used herein, “clonal” in the context of nucleic acids refers to a population of nucleic acids that comprises nucleotide sequences that are substantially or completely identical to each other at least at a given locus of interest (e.g., a target variant).

Confidence Interval: As used herein, “confidence interval” or “level of confidence” means a range of values so defined that there is a specified probability that the value of a given parameter lies within that range of values.

Copy Number Variant: As used herein, “copy number variant,” “CNV,” or “copy number variation” refers to a phenomenon in which sections of the genome are repeated and the number of repeats in the genome varies between individuals in the population under consideration.

Coverage: As used herein, “coverage” refers to the number of nucleic acid molecules that represent a particular base position.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising deoxyribonucleosides that each comprise one of four types of nucleobases, namely, adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising ribonucleosides that each comprise one of four types of nucleobases, namely, A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Detect: As used herein, “detect,” “detecting,” or “detection” refers to an act of determining the existence or presence of one or more target nucleic acids (e.g., nucleic acids having targeted mutations or other markers) in a sample.

Driver Mutation: As used herein, “driver mutation” means a mutation that drives cancer progression.

Historical Prevalence: As used herein, “historical prevalence” refers to sequence information, or data derived therefrom, obtained from one or more reference samples (e.g., from reference subjects having a given cancer type) and/or from a given subject.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-4, OX40, B7.1, B7He, LAG3, CD137, KIR, CCR5, CD27, CD40, or CD47. Other exemplary agents include proinflammatory cytokines, such as IL-1, IL-6, and TNF-α. Other exemplary agents are T-cells activated against a tumor, such as T-cells activated by expressing a chimeric antigen targeting a tumor antigen recognized by the T-cell.

Indel: As used herein, “indel” refers to mutation that involves the insertion or deletion of nucleotide positions in the genome of a subject.

LogPrior data: As used herein, “LogPrior data” refers to the log of the ratio of nucleic acid variant(s) or mutant(s) (e.g., target nucleic acid variant(s) or mutant(s)) over wild-type variants in a sample population.

MaximumMutant Allele Frequency: As used herein, “maximum mutant allele frequency,” “maximum MAF,” or “MAX MAF” refers to the maximum or largest MAF of all somatic variants present or observed in a given sample.

Mutant Allele Frequency: As used herein, “mutant allele frequency” or “MAF” refers to the frequency at which mutant alleles occur in a given population of nucleic acids, such as a sample obtained from a subject. MAF is generally expressed as a fraction or a percentage.

Mutation: As used herein, “mutation,” “variant,” or “genetic aberration” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging each different nucleic acid molecule in a given sample, or non-uniquely tagging such molecules. In the case of non-unique tagging applications, tags with a limited number of different sequences may be used to tag each nucleic acid molecule such that different molecules can be distinguished based on, for example, start and/or stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “Ali” in which the uppercase letter indicates a sample type, the Arabic numeral indicates a form of molecule within a sample, and the lowercase Roman numeral indicates a molecule within a form.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Reference Sample: As used herein, “reference sample” or “reference cfNA sample” refers a sample of known composition and/or having or known to have or lack specific properties (e.g., known nucleic acid variant(s), known cellular origin, known tumor fraction, known coverage, and/or the like) that is analyzed along with or compared to test samples in order to evaluate the accuracy of an analytical procedure. A reference sample dataset typically includes from at least about 25 to at least about 30,000 or more reference samples. In some embodiments, the reference sample dataset includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference samples.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means anything capable of being analyzed by the methods and/or systems disclosed herein.

Sensitivity: As used herein, “sensitivity” in the context of a given assay or method refers to the ability of the assay or method to detect and distinguish between targeted (e.g., nucleic acid variants) and non-targeted analytes.

Sequencing: As used herein, “sequencing” refers to any of a number of technologies used to determine the sequence (e.g., the identity and order of monomer units) of a biomolecule, e.g., a Attorney Docket No. GH0057US nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in the genome that occurs after conception. Somatic mutations can occur in any cell of the body except germ cells and accordingly, are not passed on to progeny.

Specificity: As used herein, “specificity” in the context of a diagnostic analysis or assay refers to the extent to which the analysis or assay detects an intended target analyte to the exclusion of other components of a given sample.

Sub-Clonal: As used herein, “sub-clonal” in the context of nucleic acids refers to a sub-population of nucleic acids that comprises nucleotide sequences that are substantially or completely identical to each other at least at a given locus of interest (e.g., a target variant).

Subject: As used herein, “subject” or “test subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” In some embodiments, the subject is a human who has, or is suspected of having cancer. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed with or suspected of having a disease, e.g., a cancer, an auto-immune disease.

Threshold Value: As used herein, “threshold” refers to a separately determined value used to characterize or classify experimentally determined values. In certain embodiments, for example, “threshold value” refers to a selected value to which a quantitative value is compared in order to determine that a given target nucleic acid variant is absent at a given genetic locus.

Tumor Fraction: As used herein, “tumor fraction” refers to the estimate of the fraction of nucleic acid molecules derived from tumor in a given sample. For example, the tumor fraction of a sample can be a measure derived from the maximum mutant allele frequency (MAX MAF) of the sample or coverage of the sample, or length, epigenetic state, or other properties of the cfNA fragments in the sample or any other selected feature of the sample. The term “MAX MAF” refers to the maximum or largest MAF of all somatic variants present in a given sample. In some embodiments, the tumor fraction of a sample is equal to the MAX MAF of the sample.

Value: As used herein, “value” generally refers to an entry in a dataset can be anything that characterizes the feature to which the value refers. This includes, without limitation, numbers, words or phrases, symbols (e.g., + or −) or degrees.

### DETAILED DESCRIPTION

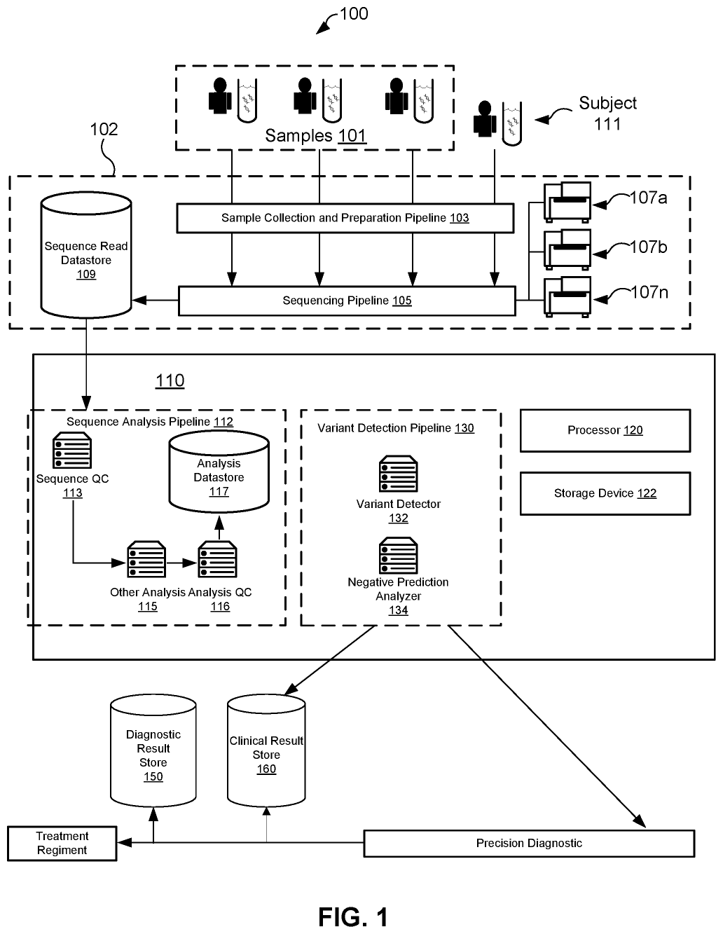

FIG. 1 illustrates an example of a system 100 for generating negative predictions of a target variant in a sample of a subject 111, according to an embodiment of the disclosure. The system 100 may process one or more samples 101 from the subject 111 to generate sequence reads for variant detection and negative predictions. The system 100 may include a laboratory system 102, a computer system 110, and/or other components. It should be noted that the laboratory system 102 and the computer system 110 may be remote from one another, and connected to one another through a computer network (not illustrated). The laboratory system 102 may include a sample collection and preparation pipeline 103, a sequencing pipeline 105, a sequence read datastore 109, and/or other components. The sequencing pipeline 105 may include one or more sequencing devices 107 (illustrated in FIG. 1 as sequencing devices 107a . . . n).

The computer system 110 may include a sequence analysis pipeline 112, a processor 120, a storage device 122, a variant detection pipeline 130, and/or other components.

The sequence analysis pipeline 112 may include a sequence quality control (QC) component 113 that may trim or trash sequence reads from the laboratory system 102, other analysis components 115 that may perform preliminary alignments to a reference genome, and an analysis QC component 116 that may perform quality control on the output of the analysis components 115. Output, such as sequence reads of a sample 101 of a subject 111, from the sequence analysis pipeline 112 may be stored in an analysis datastore 117.

Generally speaking, the processor 120 may implement (be programmed by) various components of the variant detection pipeline 130, such as the variant detector 132, the negative prediction analyzer 134, and/or other components. Alternatively, it should be noted that each of these components of the variant detection pipeline 130 may include a hardware module. Although illustrated separately for convenience, one or more of the various components or instructions, such as the variant detector 132 and the negative prediction analyzer 134 may be integrated with one another. In any event, the variant detection pipeline 130 may cause the computer system 110 to identify variants, diseases from the variants (precision diagnostics), negative predictions, and/or treatment regiments. The precision diagnostic and treatment regimen may be stored in a repository such as clinical result store 160 or diagnostic result store 150.

The variant detector 132 may determine that a target variant has not been detected based on an analysis of the sequence reads from laboratory system 102. It should be noted that at least one sequence read and/or at least one molecule that is sequenced may support the target variant—but this may not be sufficient for the variant detector 132 to detect the target variant. For instance, in some embodiments the variant detector 132 might detect the target variant only if the number of sequence reads (and/or the number of molecules that are sequenced) which support the target variant is greater than a threshold. Additionally or alternatively, the variant detector 132 might detect a target variant only if the target variant which is supported by a sequence read and/or a molecule that is sequenced meets a quality threshold. Target variants that are supported by at least one sequence read and/or at least one molecule that is sequenced, but do not meet a threshold, may thus be ignored in some embodiments as false positives, and may not be detected by the variant detector 132. Other ways to determine that a target variant has not been detected based on an analysis of the sequence reads may also be used, but further details of making this determination are omitted for clarity.

The negative prediction analyzer 134 may access the output of the variant detector 132 and confirm negative predictions as an add-on to the variant detector. Alternatively, or additionally, the negative prediction analyzer 134 may be integrated with the variant detector 132.

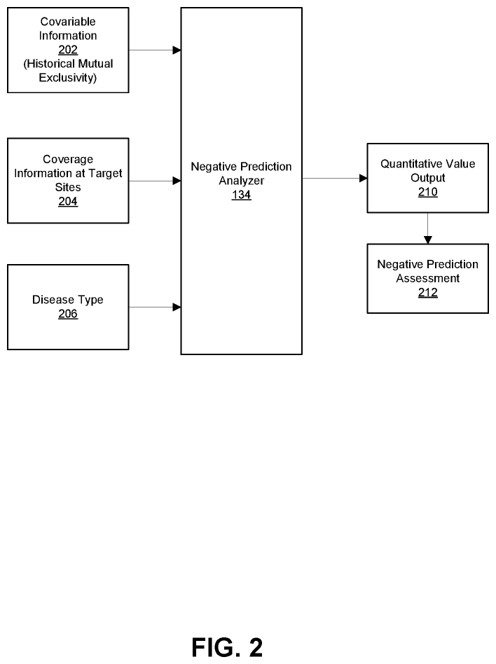

FIG. 2 illustrates a schematic diagram of exemplary inputs and outputs of a negative prediction analyzer 134, according to an embodiment. The negative prediction analyzer 134 may use covariable information 202, coverage information at target sites 204, disease type 206, and/or other input information for significance modeling. The negative prediction analyzer 134 may generate a quantitative value output 210 that may represent a likelihood of whether a negative prediction is correct and a negative prediction assessment 212 that may include a level of confidence or precision diagnostic based on the quantitative value output 210.

For example, the sequence reads from the laboratory system 102 may be aligned to a reference genome and in particular to various loci in the reference genome to determine covariable information 202. The covariable information 202 may include covariance variant information that may include historical mutual exclusivity data and/or co-occurrence data of variants. Covariable variants may refer to two or more variants that have a negative (mutually exclusive) or positive (co-occurrence) correlation to one another based on historical observations of sequence data from the laboratory system 102 and/or other data sources. For example, mutually exclusive variants may include variants that tend to not be observed with one another. Co-occurrence variants may be observed to occur when another variant is observed, such as a driver variant mutation and its co-occurrence variant.

In particular examples, the significance modeling may generate and use computational estimates of tumor fraction (TF) of a target variant based on nucleic acid sequence reads generated from the sample. Alternatively, or additionally, the significance modeling may determine and use the diversity of other variants that are detected—or not detected—in the sample. For example, the significance modeling may use detection of covariance variants that usually (based on historical covariance variant information) co-occur with the target variant or mutually exclusive variants that usually (based on the historical covariance variant information) do not co-occur with the target variant. A negative predictive value (“NPV”) may be generated based on the TF estimates and/or diversity of variants that are detected, or not detected, in the sample. The result may be used to provide a level of confidence in a negative diagnosis and/or to further guide treatment plans based on the negative diagnosis. In the context of cancer diagnosis, for example, covariance variants may include driver variants that tend to promote oncogenesis and mutually exclusive variants may include tumor suppressor variants that tend to suppress oncogenesis.

Negative Prediction

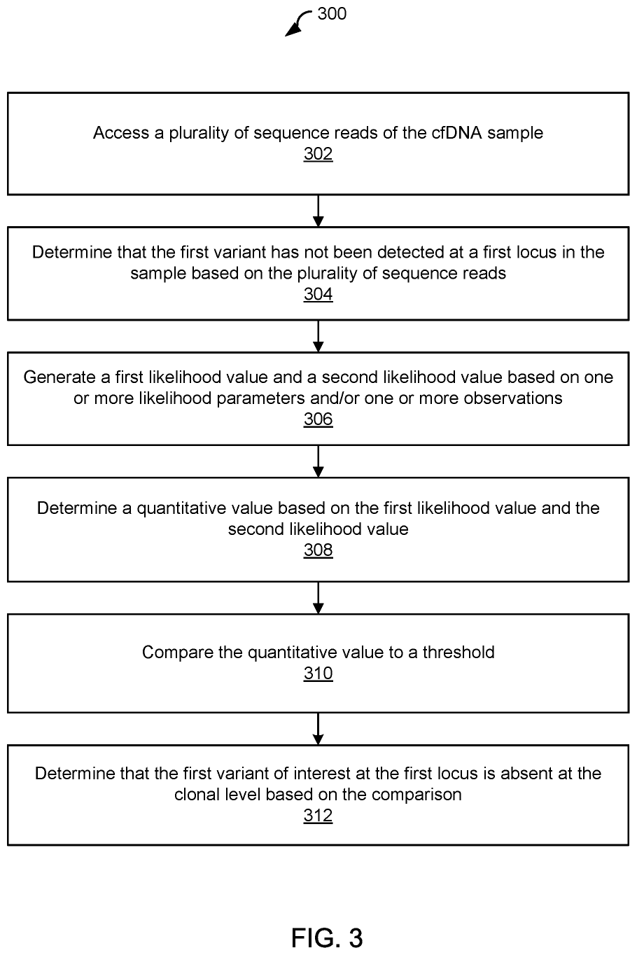

FIG. 3 illustrates an example of a method 300 for generating negative predictions of a target variant in a sample of a subject, according to an embodiment of the disclosure.

Methods of the invention can be used for determining as a true negative result that a variant of interest is absent (e.g. absent at the clonal level). Thus, with reference to FIG. 3, at 302 the method 300 may include accessing a plurality of sequence reads of the cfDNA sample. At 304, the method 300 may include determining that a target variant (the target variant) has not been detected at a first locus in the sample (e.g., a cfNA sample) based on the plurality of sequence reads. In some examples, the target variant (and/or other variants described herein) may include a somatic variant. In some examples, the target variant (and/or other variants described herein) may not include a germline variant.

Assessing Negative Predictions

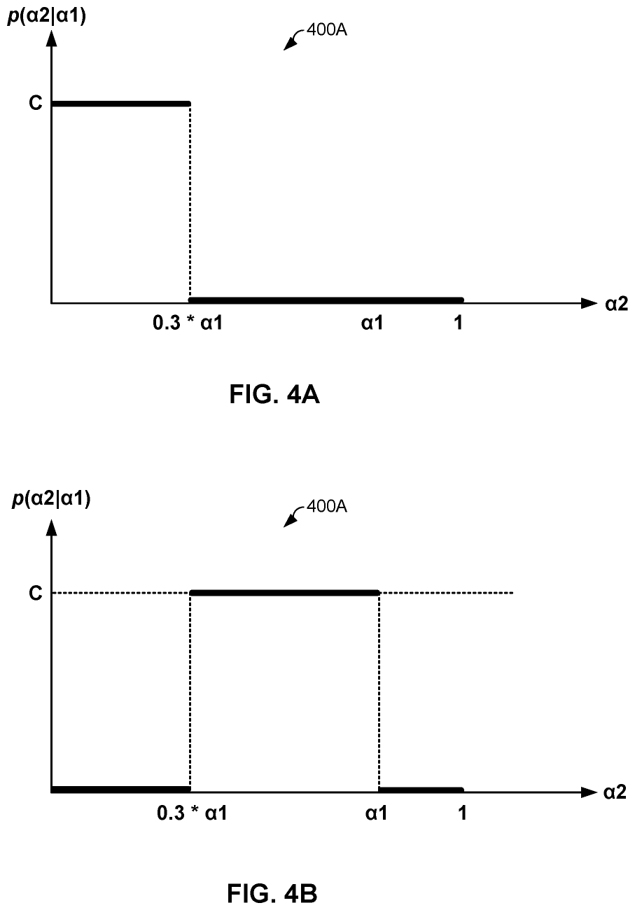

At 306, the method 300 may include generating a first likelihood value based on a probability that the target variant is absent at the clonal level and a second likelihood value based on a probability that the target variant is not absent at the clonal level. At 308, the method 300 may include determining a quantitative value based on the first likelihood value and the second likelihood value. At 310, the method 300 may include comparing the quantitative value to a threshold. At 312, the method 300 may include determining that the target variant at the first locus is absent at the clonal level based on the comparison. For example, the method 300 may include determining that the allele frequency of the target variant does not exceed the threshold (such as the sub-clonal threshold described with reference to FIGS. 4A and 4B).

Assessing Negative Predictions Based on Tumor Fraction Estimates

In some examples, the method 300 and/or the negative prediction analyzer 134 (by implementing the method 300) may model the probability that the target variant is absent at the clonal level (or present at a sub-clonal level of a tumor variant) as a test or alternative hypothesis (Hi) to generate the first likelihood value. For example, FIG. 4A illustrates a graph 400A of a test hypothesis in which a target variant (the target variant) is absent (or present at sub-clonal level of the tumor variant) from the sample, according to an embodiment. Correspondingly, the negative prediction analyzer 134 may model the probability that the target variant is not absent at the clonal level as a null hypothesis ((Ho)) to generate the second likelihood value. For example, FIG. 4B illustrates a graph 400B of a null hypothesis in which the target variant is not absent in the sample (and correlates with an allele frequency of the tumor variant), according to an embodiment. In both graphs 400A and 400B, “C” reflects the minor allele at a target locus. The value “0.3” reflects a weight applied to al (the TF estimation based on mutant allele frequency of a tumor variant) such that the product of 0.3×α1 serves as a sub-clonal threshold value. An allele frequency (α2) of a target variant in the sample 101 of the subject 111 above the sub-clonal threshold value may indicate that the target variant is correlated with the tumor variant.

In these examples, the negative prediction analyzer 134 may generate the first likelihood value and the second likelihood value by determining a tumor fraction (TF) estimate (such as α₁ in the Equations described herein) of the sample. The TF estimate may indicate a fraction of tumor DNA detected in the sample. In some examples, the TF estimate may be determined by determining an allele frequency of a tumor variant (referred to as MAX MAF) in the sample. The MAX MAF may be determined by determining a molecule count associated with the tumor variant based on the plurality of sequence reads. The first likelihood value based on the probability that the target variant is absent at the clonal level (such as L₁ in the Equations described herein) and the second likelihood value that the target variant is not absent at the clonal level or is present at a sub-clonal level (such as L₀ in the Equations described herein) may be based on the TF estimate.

In some embodiments, the negative prediction analyzer 134 may use the TF estimate to generate the quantitative value that assesses the quality of the negative prediction (such as by indicating a probability of whether or not the negative prediction is correct or false). For example, the negative prediction analyzer 134 may determine a first allele frequency of the target variant (the target variant). The negative prediction analyzer 134 may determine the first allele frequency by determining a first molecule count associated with the target variant based on the plurality of sequence reads. The negative prediction analyzer 134 may use the first allele frequency with the MAX MAF to determine the first likelihood value and the second likelihood value are based further on the first allele frequency and the MAX MAF.

Referring to FIG. 4A, the probability that the target variant is absent at the clonal level (or present at a sub-clonal level) may be based on a sub-clonal threshold value (illustrated as 0.3*α1). Which may be a sub-clonal weight (illustrated as 0.3) multiplied by a tumor fraction estimate (illustrated as an allele frequency such as MAX MAF of a tumor variant). The sub-clonal threshold value may be determined based on specific genes, cancer type, or other expected values. These values may range anywhere from 0.01 to 0.99, including but not limited to 0.01, 0.10, 0.20, 0.30, 0.40, 0.50, 0.60, 0.70, 0.80, 0.90, and 0.99. Equations 1-3 that follow relate to generating the first and second likelihood values and resulting quantitative value in certain embodiments.

L₁=∫_{α}_{₁}∫_{α}_{₂}P(Mᵥ,Mᵣ|α₁,ε)*P(Mᵥ′,Mᵣ′|α₂,ε′)*p(α₁,α₂)dα₁dα₂ (Eq. 1)

p(α₁,α₂)=p(α₁)*p(α₂|α₁) (Eq. 2)

∫₀¹p(α₂|α₁)dα₂=1,∀α₁ (Eq. 3)

(sum of probabilities for all possible values)

Referring to Eqs. 1-3,
L₁ refers to the likelihood value for the test hypothesis where the variant is absent at the clonal level. Null hypothesis generated using the same formula for L₁, but alpha 2 has a different range of values (e.g., 0.3 to 1).
α₁ refers to an allele frequency of a tumor variant, which may be used as a TF estimate
α₂ refers to an allele frequency of a target variant (the target variant)
Mᵥ refers to a number of molecules supporting a tumor variant at a locus of the tumor variant
Mᵣ refers to a number of molecules supporting a reference wildtype at the locus of the tumor variant
Mᵥ′ refers to a number of molecules supporting a target variant at a locus of the target variant
Mr′ refers to a number of molecules supporting a reference wildtype at the locus of the target variant
ε refers to an error rate for the TF estimate
ε′ refers to an error rate for the target variant
Error rates are typically derived from sequence information obtained from samples obtained from healthy or normal subjects (e.g., z-scores or the like).

α₂=t*α₁ (Eq. 4) This equation is for simplification purposes (same as for Eq. 1), but is easier to compute than the integral in Eq. 1.

∈ and ∈ error rates in tumor fraction (maxmaf) and target variants correspondingly

$$\begin{matrix}\frac{1}{1 - 0.3} {\int }_{0}^{1} {( ( 1 - {α}_{1} ) * ( 1 - ϵ ) )}^{{M}_{r}} * {( {α}_{1} + ( 1 - {α}_{1} ) ϵ )}^{{M}_{v}} * {\int }_{0.3}^{1} {( ( 1 - t * {α}_{1} ) * ( 1 - {ϵ}^{′} ) )}^{{M}_{r}^{′}} * {( t * {α}_{1} + ( 1 - t * {α}_{1} ) {ϵ}^{′} )}^{{M}_{v}^{′}} {dtdα}_{1} & ( Eq . 5 ) \\ {\frac{1}{0.3 - 0} {\int }_{0}^{1} {( ( 1 - {α}_{1} ) * ( 1 - ϵ ) )}^{{M}_{r}} * {α}_{1} + ( 1 - {α}_{1} ) ϵ )}^{{M}_{v}} * {\int }_{0.0}^{0.3} {( ( 1 - t * {α}_{1} ) * ( 1 - {ϵ}^{′} ) )}^{{M}_{r}^{′}} * {( t * {α}_{1} + ( 1 - t * {α}_{1} ) {ϵ}^{′} )}^{{M}_{v}^{′}} {dtdα}_{1} & ( Eq . 6 )\end{matrix}$$

Epsilon (∈) is taken from calculation of a z-score derived from sequence information obtained from samples obtained from healthy or normal subjects.

In the equations that follow:

T⁻ refers to the target variant is absent on clonal level

T⁺ refers to target variant is present on clonal level

Vᵢ⁺ refers to variant (other than target) is present (i=1, . . . , n all other called variants)

[symbol] ᵢ refers to likelihood value (base hypothesis i=0, test hypothesis i=1)

Adjusting the Quantitative Value Based on Prevalence of Other Variants

In some examples, the negative prediction analyzer 134 may adjust the quantitative value determined from the TF estimate based on the presence of one or more variants other than the target variant in a sample 101 of the subject 111. For example, the negative prediction analyzer 134 may determine a prevalence of at least a second variant in the cfDNA sample 101, and adjust the quantitative value based on the prevalence of at least a second variant.

For example, the prevalence data may be determined according to Equations 7 and 8:

$$\begin{matrix}P ( {T}^{-} \vert {V}_{i}^{+} ) = \frac{P ( {T}^{-} , {V}_{i}^{+} )}{P ( {V}_{i}^{+} )} = \frac{P ( {T}^{-} , {V}_{i}^{+} )}{P ( {T}^{-} , {V}_{i}^{+} ) + P ( {T}^{+} , {V}_{i}^{+} )} = \frac{{c}_{i} ( F , T )}{{c}_{i} ( F , T ) + {c}_{i} ( T , T )} & ( Eq . 7 ) \\ P ( {T}^{+} \vert {V}_{i}^{+} ) = \frac{P ( {T}^{+} , {V}_{i}^{+} )}{P ( {V}_{i}^{+} )} = \frac{P ( {T}^{+} , {V}_{i}^{+} )}{P ( {T}^{+} , {V}_{i}^{+} ) + P ( {T}^{-} , {V}_{i}^{+} )} = \frac{{c}_{i} ( T , T )}{{c}_{i} ( T , T ) + {c}_{i} ( F , T )} & ( Eq . 8 )\end{matrix}$$

The likelihood value (L1) that the test hypothesis is correct may be adjusted based on Equation 9 to generate an adjusted likelihood value (L₁ₐ), and a likelihood ratio (LRₐ) may be generated according to Equation 10:

$$\begin{matrix} {ℒ}_{1 a} = {ℒ}_{1} * \frac{P ( {T}^{-} \vert {V}_{i}^{+} , i ∈ \{ 1 , . . . , n \} )}{P ( {T}^{-} )} & ( Eq . 9 ) \\ L {R}_{a} = \frac{{ℒ}_{1 a}}{{ℒ}_{0 a}} = L R * \frac{P ( {T}^{-} \vert {V}_{i}^{+} , i ∈ \{ 1 , . . . , n \} )}{P ( {T}^{+} \vert {V}_{i}^{+} , i ∈ \{ 1 , . . . , n \} )} * \frac{P ( {T}^{+} )}{P ( {T}^{-} )} = L R * \frac{{Π}_{i = \overline{1 , n}} P ( {T}^{-} \vert {V}_{i}^{+} )}{{Π}_{i = \overline{1 , n}} P ( {T}^{+} \vert {V}_{i}^{+} )} * \frac{{P ( {T}^{+} )}^{n}}{{P ( {T}^{-} )}^{n}} & ( Eq . 10 )\end{matrix}$$

Eq. 10 is a likelihood ratio using the properties of condition dependence.

Assessing Negative Predictions Based on LLRs

In some examples, the quantitative value may be based on an LLR between the first likelihood value and the second likelihood value. As such, the quantitative value may be based on a ratio between the first likelihood value (such as L₁ of Equation 14) and the second likelihood value (such as L₀ of Equation 15). In some examples, the negative prediction analyzer 134 may generate a TF-based LLR (such as LLR_{tf} illustrated in Equation 16). The negative prediction analyzer 134 may generate the quantitative value (such as LLR) based on Equation 11:

LLR=LLR_{tf}+LLRₘₑ (Eq. 11)

(Log likelihood ratio (LLR) of tumor fraction (LLRₜf) and mutual exclusivity (LLRₘₑ).

Assessing Negative Predictions Using LLR Based on Covariance (Mutual Exclusivity) Data

In some examples, the quantitative value may be based on LLR of covariance data. For example, the negative prediction analyzer 134 may generate the LLRₘₑ that reflects covariance data, as illustrated in Equation 18 (conditional probability of how many times variants are observed together.

$$\begin{matrix}P ( {V}_{i}^{+} \vert {T}^{+} ) = \frac{P ( {V}_{i}^{+} , {T}^{+} )}{P ( {T}^{+} )} = \frac{P ( {V}_{i}^{+} , {T}^{+} )}{P ( {V}_{i}^{+} , {T}^{+} ) + P ( {V}_{i}^{-} , {T}^{+} )} = \frac{{c}_{i} ( T , T )}{{c}_{i} ( T , T ) + {c}_{i} ( T , F )} & ( Eq . 12 ) \\ P ( {V}_{i}^{+} \vert {T}^{-} ) = \frac{{c}_{i} ( F , T )}{{c}_{i} ( F , T ) + {c}_{i} ( F , F )} & ( Eq . 13 )\end{matrix}$$

Assessing Negative Predictions Using Combinations of LLRs

In some embodiments, the quantitative value may be expressed as a log posterior probability ratio (LPPR) based on a combination of the TF-based log likelihood of whether the null or test hypothesis is correct, a covariance-based (e.g., mutual exclusivity) log likelihood of whether the null or test hypothesis is correct, and prior-data based log data, such as expressed in Equations 19 and 21 below. In some examples, the quantitative value (such as an LLR in Equation 11) may be based further on a LogPrior data that is based on historical, observed, data not necessarily limited to the sample 101 of the subject 111. Such LogPrior data may be based on covariable information indicating a historical prevalence of one or more variants exhibiting co-occurrence and/or mutual exclusivity with the target variant. For example, the LogPrior data may be expressed as:

$$log \frac{P ( {T}^{-} )}{P ( {T}^{+} )} .$$

The LogPrior data may be used to generate the quantitative value in combination with other values, such as in Equation 19.

$$\begin{matrix} {ℒ}_{1} = P ( {M}_{v} , {M}_{r} \vert {T}^{-} ) & ( Eq . 14 ) \\ {ℒ}_{0} = P ( {M}_{v} , {M}_{r} \vert {T}^{+} ) & ( Eq . 15 ) \\ {LLR}_{t f} = log \frac{{ℒ}_{1}}{{ℒ}_{0}} = log \frac{P ( {M}_{v} , {M}_{r} \vert {T}^{-} )}{P ( {M}_{v} , {M}_{r} \vert {T}^{+} )} & ( Eq . 16 ) \\ LL {R}_{m e} = log \frac{P ( {V}_{i}^{+} , i = \{ 1 , . . . , n \} \vert {T}^{-} )}{P ( {V}_{i}^{+} , i = \{ 1 , . . . , n \} \vert {T}^{+} )} = {\sum }_{i = \{ 1 , . . . , n \}} log \frac{P ( {V}_{i}^{+} \vert {T}^{-} )}{P ( {V}_{i}^{+} \vert {T}^{+} )} & ( Eq . 17 ) \\ LLR = L L {R}_{t f} + L L {R}_{m e} = log \frac{P ( {M}_{v} , {M}_{r} \vert {T}^{-} )}{P ( {M}_{v} , {M}_{r} \vert {T}^{+} )} + {\sum }_{i = \{ 1 , . . . , n \}} ( log P ( {V}_{i}^{+} \vert {T}^{-} ) - log P ( {V}_{i}^{+} \vert {T}^{+} ) ) & ( Eq . 18 ) \\ log \frac{P ( {T}^{-} \vert O , V )}{P ( {T}^{+} \vert O , V )} = log \frac{P ( O \vert {T}^{-} )}{P ( O \vert {T}^{+} )} + log \frac{P ( V \vert {T}^{-} )}{P ( V \vert {T}^{+} )} + log \frac{P ( {T}^{-} )}{P ( {T}^{+} )} & ( Eq . 19 )\end{matrix}$$

LPPR=TF-based+Covariance-based+Prior-data based

$$\begin{matrix} log Prior = log \frac{P ( {T}^{-} )}{P ( {T}^{+} )} & ( Eq . 20 ) \\ LPPR = log \frac{P ( {T}^{-} \vert O , V )}{P ( {T}^{+} \vert O , V )} = LL {R}_{tf} + L L {R}_{m e} + log Prior = log \frac{P ( {M}_{v} , {M}_{r} \vert {T}^{-} )}{P ( {M}_{v} , {M}_{r} \vert {T}^{+} )} + {\sum }_{i = \{ 1 , . . . , n \}} ( log P ( {V}_{i}^{+} \vert {T}^{-} ) - log P ( {V}_{i}^{+} \vert {T}^{+} ) ) + log P ( {T}^{-} ) - log P ( {T}^{+} ) & ( Eq . 21 ) \\ P ( {T}^{-} \vert O , V ) = \frac{1}{{e}^{- L P P R} + 1} . & ( Eq . 22 )\end{matrix}$$

It should be understood that in the previous examples, the negative prediction analyzer 134 has been described as implementing the method 300 and performing the foregoing additional operations. It should be further understood that the foregoing additional operations may be part of and extend the method 300.

The various processing operations and/or methods depicted in the Figures may be accomplished using some or all of the system components described in detail herein and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail herein) are provided as example and, as such, should not be viewed as limiting.

Computer Implementation

The present methods can be computer-implemented, such that any or all of the operations described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The present disclosure provides computer control systems that are programmed to implement methods of the disclosure. The processor 120 may include a single core or multi core processor, or a plurality of processors for parallel processing. The storage device 122 may include random-access memory, read-only memory, flash memory, a hard disk, and/or other type of storage. The computer system 110 may include a communication interface (e.g., network adapter) for communicating with one or more other systems, and peripheral devices, such as cache, other memory, data storage and/or electronic display adapters. The components of the computer system 110 may communicate with one another through an internal communication bus, such as a motherboard. The storage device 122 may be a data storage unit (or data repository) for storing data. The computer system 110 may be operatively coupled to a computer network (“network”) with the aid of the communication interface. The network may be the Internet, an internet and/or extranet, or an intranet and/or extranet that is in communication with the Internet. The network in some cases is a telecommunication and/or data network. The network may include a local area network. The network may include one or more computer servers, which can enable distributed computing, such as cloud computing. The network, in some cases with the aid of the computer system 110, may implement a peer-to-peer network, which may enable devices coupled to the computer system 120 to behave as a client or a server.

The processor 120 may execute a sequence of machine-readable instructions, which can be embodied in a program or software. The instructions may be stored in a memory location, such as the storage device 122. The instructions can be directed to the processor 120, which can subsequently program or otherwise configure the processor 120 to implement methods of the present disclosure. Examples of operations performed by the processor 120 may include fetch, decode, execute, and writeback.

The processor 120 may be part of a circuit, such as an integrated circuit. One or more other components of the system 100 may be included in the circuit. In some cases, the circuit may include an application specific integrated circuit (ASIC).

The storage device 122 may store files, such as drivers, libraries and saved programs. The storage device 122 can store user data, e.g., user preferences and user programs. The computer system 110 in some cases may include one or more additional data storage units that are external to the computer system 110, such as located on a remote server that is in communication with the computer system 110 through an intranet or the Internet.

The computer system 110 can communicate with one or more remote computer systems through the network. For instance, the computer system 110 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 110 via the network.

Methods as described herein can be implemented by way of machine (e.g., computer processor) executable code stored on an electronic storage location of the computer system 110, such as, for example, on the storage device 122. The machine executable or machine readable code can be provided in the form of software (e.g., computer readable media). During use, the code can be executed by the processor 120. In some cases, the code can be retrieved from the storage device 122 and stored on the storage device 122 for ready access by the processor 120.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 110, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 110 can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 120.

Sample Collection and Analysis Pipeline

A sample 101 may be any biological sample isolated from a subject. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double- and/or single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA).

In certain embodiments, the polynucleotides can be enriched prior to sequencing. Enrichment can be performed for specific target regions (“target sequences”) or nonspecifically. In some embodiments, targeted regions of interest may be enriched with capture probes (“baits”) selected for one or more bait set panels using a differential tiling and capture scheme. A differential tiling and capture scheme uses bait sets of different relative concentrations to differentially tile (e.g., at different “resolutions”) across genomic regions associated with baits, subject to a set of constraints (e.g., sequencer constraints such as sequencing load, utility of each bait, etc.), and capture them at a desired level for downstream sequencing. These targeted genomic regions of interest may include regions of a subject's genome or transcriptome. In some embodiments, biotin-labeled beads with probes to one or more regions of interest can be used to capture target sequences, optionally followed by amplification of those regions, to enrich for the regions of interest.

Sequence capture typically involves the use of oligonucleotide probes that hybridize to the target sequence. A probe set strategy can involve tiling the probes across a region of interest. Such probes can be, e.g., about 60 to 130 bases long. The set can have a depth of about 2×, 3×, 4×, 5×, 6×, 8×, 9×, 10×, 15×, 30×, 50×, or more. The effectiveness of sequence capture depends, in part, on the length of the sequence in the target molecule that is complementary (or nearly complementary) to the sequence of the probe.

In some embodiments, the methods of the disclosure comprise selectively enriching regions from the subject's genome or transcriptome prior to sequencing. In other embodiments, the methods of the disclosure comprise non-selectively enriching regions from the subject's genome or transcriptome prior to sequencing.

In certain embodiments, sample index sequences are introduced to the polynucleotides after enrichment. The sample index sequences may be introduced through PCR or ligated to the polynucleotides, optionally as part of adapters.

The volume of plasma can depend on the desired read depth for sequenced regions. Exemplary volumes are 0.4-40 ml, 5-20 ml, 10-20 ml. For example, the volume can be 0.5 ml, 1 ml, 5 ml, 10 ml, 20 ml, 30 ml, or 40 ml. A volume of sampled plasma may be 5 to 20 ml.

The sample can comprise various amounts of nucleic acid that contains genome equivalents. For example, a sample of about 30 ng DNA can contain about 10,000 (104) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell free. A sample can comprise nucleic acids carrying mutations. For example, a sample can comprise DNA carrying germline mutations and/or somatic mutations. A sample can comprise DNA carrying cancer-associated mutations (e.g., cancer-associated somatic mutations).

Exemplary amounts of cell free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids have an exemplary size distribution of about 100-500 nucleotides, with molecules of 110 to about 230 nucleotides representing about 90% of molecules, with a mode of about 168 nucleotides in humans and a second minor peak in a range between 240 to 430 nucleotides. Cell-free nucleic acids can be about 160 to about 180 nucleotides, or about 320 to about 360 nucleotides, or about 430 to about 480 nucleotides.

Cell-free nucleic acids can be isolated from bodily fluids through a partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, cell-free nucleic acids can be precipitated with an alcohol. Further clean up steps may be used such as silica based columns to remove contaminants or salts. Non-specific bulk carrier nucleic acids, for example, may be added throughout the reaction to optimize certain aspects of the procedure such as yield.

After such processing, samples can include various forms of nucleic acid including double-stranded DNA, single stranded DNA and single stranded RNA. Optionally, single stranded DNA and RNA can be converted to double-stranded forms so they are included in subsequent processing and analysis steps.

### Amplification

Sample nucleic acids flanked by adapters can be amplified by PCR and other amplification methods typically primed from primers binding to primer binding sites in adapters flanking a DNA molecule to be amplified. Amplification methods can involve cycles of extension, denaturation and annealing resulting from thermocycling or can be isothermal as in transcription mediated amplification. Other amplification methods include the ligase chain reaction, strand displacement amplification, nucleic acid sequence-based amplification, and self-sustained sequence-based replication.

One or more amplifications can be applied to introduce barcodes to a nucleic acid molecule using conventional nucleic acid amplification methods. The amplification can be conducted in one or more reaction mixtures. Molecule tags and sample indexes/tags can be introduced simultaneously, or in any sequential order. Molecule tags and sample indexes/tags can be introduced prior to and/or after sequence capturing. In some cases, only the molecule tags are introduced prior to probe capturing while the sample indexes/tags are introduced after sequence capturing. In some cases, both the molecule tags and the sample indexes/tags are introduced prior to probe capturing. In some cases, the sample indexes/tags are introduced after sequence capturing. Usually, sequence capturing involves introducing a single-stranded nucleic acid molecule complementary to a targeted sequence, e.g., a coding sequence of a genomic region and mutation of such region is associated with a cancer type. Typically, the amplifications generate a plurality of non-uniquely or uniquely tagged nucleic acid amplicons with molecule tags and sample indexes/tags at a size ranging from 200 nt to 700 nt, 250 nt to 350 nt, or 320 nt to 550 nt. In some embodiments, the amplicons have a size of about 300 nt. In some embodiments, the amplicons have a size of about 500 nt.

### Barcodes

Barcodes can be incorporated into or otherwise joined to adapters by chemical synthesis, ligation, overlap extension PCR among other methods. Generally, assignment of unique or non-unique barcodes in reactions follows methods and systems described by US patent applications 20010053519, 20110160078, and U.S. Pat. Nos. 6,582,908 and 7,537,898 and 9,598,731.

Tags can be linked to sample nucleic acids randomly or non-randomly. In some cases, they are introduced at an expected ratio of identifiers (i.e., a combination of barcodes) to microwells. The collection of barcodes can be unique, e.g., all the barcodes have a different nucleotide sequence. The collection of barcodes can be non-unique, i.e., some of the barcodes have the same nucleotide sequence, and some of the barcodes have different nucleotide sequence. For example, the identifiers may be loaded so that more than 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the identifiers may be loaded so that less than 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample.

A preferred format uses 20-50 different tags, ligated to both ends of a target molecule creating 20-50×20-50 tags, i.e., 400-2500 tag combinations. Such numbers of tags are sufficient that different molecules having the same start and stop points have a high probability (e.g., at least 94%, 99.5%, 99.99%, 99.999%) of receiving different combinations of tags.

In some cases, identifiers may be predetermined or random or semi-random sequence oligonucleotides. In other cases, a plurality of barcodes may be used such that barcodes are not necessarily unique to one another in the plurality. In this example, barcodes may be attached (e.g., by ligation or PCR amplification) to individual molecules such that the combination of the barcode and the sequence it may be attached to creates a unique sequence that may be individually tracked. As described herein, detection of non-uniquely tagged barcodes in combination with beginning (start) and/or end (stop) genomic coordinates of a given sequenced sample molecule (i.e., excluding sequence information obtained from the barcodes, adaptors, and the like)) may allow assignment of a unique identity to a particular molecule. The length, or number of base pairs, of an individual sequenced sample molecule (i.e., exclusive of sequence information corresponding to barcodes, adaptors, and the like) may also be used to assign a unique identity to such a molecule. As described herein, fragments from a single strand of nucleic acid having been assigned a unique identity, may thereby permit subsequent identification of fragments from the parent strand, and/or a complementary strand.

### Sequencing Pineline

Sample nucleic acids flanked by adapters with or without prior amplification can be subject to sequencing, such as by one or more sequencing devices 107. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may be multiple lanes, multiple channels, multiple wells, or other means of processing multiple sample sets substantially simultaneously. Sample processing unit can also include multiple sample chambers to enable processing of multiple runs simultaneously.

The sequencing reactions can be performed on one or more fragments types known to contain markers of cancer of other disease. The sequencing reactions can also be performed on any nucleic acid fragments present in the sample. The sequence reactions may provide for sequencing at least 5%, 10%, 15%, 20%, 25%, 30%, 40%, 50%, 60%, 70%, 80%, 90%, 95%, 99%, 99.9% or 100% of a given genome. In other cases, the sequence reactions may provide for sequencing less than 5%, 10%, 15%, 20%, 25%, 30%, 40%, 50%, 60%, 70%, 80%, 90%, 95%, 99%, 99.9% or 100% of a given genome.

Simultaneous sequencing reactions may be performed using multiplex sequencing. In some cases, cell free polynucleotides may be sequenced with at least 1000, 2000, 3000, 4000, 5000, 6000, 7000, 8000, 9000, 10000, 50000, 100,000 sequencing reactions. In other cases, cell free polynucleotides may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

Sequence Analysis Pipeline

The present methods can be used to diagnose the presence or absence of conditions, particularly cancer, in a subject, to characterize conditions (e.g., staging cancer or determining heterogeneity of a cancer), monitor response to treatment of a condition, effect prognosis risk of developing a condition or subsequent course of a condition.

Various cancers may be detected using the present methods. Cancer cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancer in individuals using the methods and systems described herein.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

Cancers can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive or dormant. The system and methods of this disclosure may be useful in determining disease progression.

The present analysis is also useful in determining the efficacy of a particular treatment option. Successful treatment options may increase the amount of copy number variation or rare mutations detected in a subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens are changing during the course of infection. This may be particularly important during chronic infections, such as HIV/AIDs or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject, the method comprising generating a genetic profile of extracellular polynucleotides in the subject, wherein the genetic profile comprises a plurality of data resulting from copy number variation and rare mutation analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and rare mutation analyses alone or in combination.

The present methods can be used to diagnose, prognose, monitor or observe cancers or other diseases of fetal origin. That is, these methodologies may be employed in a pregnant subject to diagnose, prognose, monitor or observe cancers or other diseases in an unborn subject whose DNA and other polynucleotides may co-circulate with maternal molecules.

Exemplary Precision Treatments

The precision diagnostics provided by the improved computer system 110 may result in precision treatment plans, which may be identified by the computer system 110 (and/or curated by health professionals). For example, in lung cancer and other diseases, a goal may be to ensure that no superior treatment options exist, given presence of a given variant. For example, EGFR (L858R, exon 19 deletion), BRAF V600E, ALK, and ROS1 fusions may be treated with targeted therapies that may be more suitable than platinum- and chemo-therapies. Although these are examples of the primary drivers, other targetable drivers exist, such as MET exon 14 skipping. In another example, for colon cancer, the goal may be to avoid non-effective treatments. Chemotherapy with FOLFIRI or Chemotherapy with irinotecan regimens may be supplemented with Cetuximab or Panitumumab if KRAS or NRAS is wildtype. Thus, confidence in whether KRAS and NRAS are wildtype will increase confidence that adding Cetuximab or Panitumumab is the correct treatment option and no further testing may be required. The biological explanation for this is that Cetuximab or Panitumumab target EGFR and inhibit its activity. RAS (K/NRAS) is downstream of EGFR, so if RAS is activated, inhibiting EGFR will have minimal or no impact, so the Cetuximab or Panitumumab treatment will be administered inappropriately.

As additional therapies are developed for various diseases, interpretation of negative prediction will become increasingly complicated but critical in designing precision therapies.

Another goal may be to guide whether a downstream diagnostic procedure is performed. For instance, by determining the absence of a variant, it may be possible to avoid (or to recommend to avoid) an expensive or invasive diagnostic test e.g. an imaging procedure, a scan (such as a CT, MRI or PET scan), an endoscopic procedure, and/or a solid tissue biopsy (such as a needle biopsy). It may also be possible to avoid (or to recommend to avoid) another liquid biopsy test (e.g., blood, plasma, urine, cerebrospinal fluid) or stool test. Results based on a blood assay may thus be used to guide reflex tissue testing and to avoid the need for a solid tissue biopsy to confirm the wild-type status for any potential variant of interest. Negative predictions as described above may be used to assess the probability of absence of a clinically significant mutation in a liquid biopsy, which may give confidence that the liquid biopsy was sufficient for detecting the potential presence of a variant of interest, and that a downstream diagnostic procedure is not needed. This may also facilitate timely therapeutic decision making.

Nucleotide variations in sequenced nucleic acids can be determined by comparing sequenced nucleic acids with a reference sequence. The reference sequence is often a known sequence, e.g., a known whole or partial genome sequence from an object, whole genome sequence of a human object. The reference sequence can be hG19. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeds a threshold, then a variant nucleotide can be called at the designated position. The threshold can be a simple number, such as at least 1, 2, 3, 4, 5, 6, 7, 8, 9, or 10 sequenced nucleic acid within the subset including the nucleotide variant or it can be a ratio, such as a least 0.5, 1, 2, 3, 4, 5, 10, 15, or 20 of sequenced nucleic acids within the subset include the nucleotide variant, among other possibilities. The comparison can be repeated for any designated position of interest in the reference sequence. Sometimes a comparison can be performed for designated positions occupying at least 20, 100, 200, or 300 contiguous positions on a reference sequence, e.g., 20-500, or 50-300 contiguous positions.

### EXAMPLES

### Example 1: Liquid Biopsy Wild Type Prediction of Negative Predictors for Anti-EGFR Therapy in Advanced Colorectal Cancer CRC

Methods

An analytical method was developed for the Guardant360@ ctDNA test (Guardant Health, Redwood City, Calif.) that jointly analyzes the estimated tumor fraction and the presence of mutually exclusive mutations to provide a yes/no/unevaluable wild-type status for clonal activating RAS/RAF mutations.

Results

To validate this method and the model's confidence in the clonal wild-type determination, a subset of samples from patients with CRC and known positive RAS/RAF mutation status by tissue sequencing who underwent clinical Guardant360 testing (n=98) was used. 79 had concordant detection of RAS/RAF while 19 had no detected RAS/RAF mutation by Guardant360, which could be used to confirm the model's predictions. The model correctly identified all 19 samples as unevaluable for wild type status and did not provide high confidence wild-type calls in the presence of known RAS/RAF mutations. To assess overall performance, this method was applied to a cohort of samples from over 8,500 patients with CRC and were able to make high confidence determination of either RAS/RAF mutant (40.7%) or clonal wild-type status (21.3%), significantly expanding the cohort of patients for whom final determination of the RAS/RAF status could be reliably achieved through ctDNA testing.

Conclusions

Guardant360 ctDNA testing can reliably determine wild-type status of RAS/RAF genes in the majority of advanced CRC patients and reliably guide anti-EGFR therapy decisions.

### Example 2: Mutual Exclusivity and Mutational Co-Occurrence Observed in Advanced Cancer Liquid Biopsy

Introduction

Somatic mutations in patients with treatment-naive solid tumors tend to be clonal and frequently display histology specific stereotyped patterns of mutational occurrence. For example, in patients with treatment naive non-small cell lung cancer (NSCLC), EGFR exon 19 deletions have not been observed to co-occur with other driver mutations such as MET exon 14 skipping deletions or EML4-ALK fusions (TCGA, 2017). In contrast, tumors from patients with previously treated disease have been subjected to different biological and drug environments that influence their tumor biology and mutational patterns. Using the Guardant360 cell-free circulating tumor DNA (ctDNA) plasma test we characterized mutational patterns in very large cohorts of advanced NSCLC and colorectal (CRC) cancers.

Methods

De-identified results from patients with advanced NSCLC (n=59,589) and CRC (n=13,116) who underwent clinical Guardant360 testing (Guardant Health, Redwood City, Calif.) were used for the analysis of mutual exclusivity and co-occurrence of variants. Patients were both treatment naive and previously treated. Variants included in the analysis required at least 200 observations, each with a variant allele fraction greater than 0.01. Variants that met criteria were assessed using Fisher's exact test and corrected for multiple tests with the Bonferroni method.

Results

In over 59,000 ctDNA results from patients with advanced NSCLC, previously reported tissue analysis findings of the mutual exclusivity of known NSCLC drivers such as EGFR exon 19 deletions with MET exon 14 skipping alterations were confirmed. An additional 70 pairs of mutually exclusive mutations was discovered including novel pairs with mutations in STKI1, TERT, and BRAF (class 3) observed to be mutually exclusive with known NSCLC driver mutations. Also observed were exclusive co-occurrence of EGFR resistance mutations T790M and C797S with EGFR drivers, recapitulating the co-occurrence seen in the TCGA. In CRC, a cancer type not classically known for mutually exclusive driver mutations, analysis of over 13,000 cases identified previously undescribed mutually exclusivity between variants BRAF V600E and APC R876*, p<0.005. Additional pairs of specific mutually exclusive mutations were found in KRAS, BRAF, APC, and TP53.

Conclusions

Utilizing very large advanced NSCLC and CRC cohorts tested with ctDNA plasma-based comprehensive genomic profiling, previously reported patterns of mutually exclusive driver mutations were confirmed and novel patterns of co-occurrence and exclusivity were discovered. These results highlight the utility of ctDNA for the identification of clinically relevant mutations and novel biological mutational patterns.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

1. A method of determining that a first variant of interest at a first locus is absent at a clonal level in a cell-free deoxyribonucleic acid (cfDNA) sample of a human subject, the method comprising:
accessing a plurality of sequence reads of the cfDNA sample;
determining that the first variant has not been detected at the first locus in the sample based on the plurality of sequence reads;
generating a first likelihood value based on a probability that the first variant is absent at the clonal level and a second likelihood value based on a probability that the first variant is not absent at the clonal level;
determining a quantitative value based on the first likelihood value and the second likelihood value;
comparing the quantitative value, the first likelihood value, and the second likelihood value to a threshold; and
determining that the first variant of interest at the first locus is absent at the clonal level based on the comparison.

2. The method of claim 1, wherein generating the first likelihood value and the second likelihood value comprises:
determining a tumor fraction estimate of the sample, wherein the first likelihood value and the second likelihood value is based on the tumor fraction estimate.

3. The method of claim 2, wherein determining the tumor fraction estimate comprises:
determining a maximum mutant allele frequency (MAX MAF) of a tumor mutation in the sample.

4. The method of claim 3, wherein determining the MAX MAF comprises determining a molecule count associated with the tumor mutation based on the plurality of sequence reads.

5. The method of claim 3, wherein generating the first likelihood value and the second likelihood value comprises:
determining an allele frequency of at least a second variant, wherein the first likelihood value and the second likelihood value are based further on the allele frequency and the MAX MAF.

6. The method of claim 5, further comprising:
comparing the allele frequency with a second threshold that is based on the MAX MAF, wherein determining that the first variant of interest at the first locus is absent at the clonal level is based further on the comparison of the MAF with the second threshold.

7. The method of claim 5, wherein determining the allele frequency comprises:
determining a first molecule count associated with the first variant based on the plurality of sequence reads.

8. The method of claim 5, wherein determining the quantitative value comprises:
accessing covariable information indicating a historical prevalence of one or more variants exhibiting co-occurrence and/or mutual exclusivity with the first variant, wherein the quantitative value is based on the covariable information.

9. The method of claim 8, further comprising:
determining a prevalence of at least a second variant in the cfDNA sample, wherein the quantitative value is based further on the covariable information.

10. The method of claim 1, wherein determining the quantitative value comprises:
accessing covariable information indicating a historical prevalence of one or more variants exhibiting co-occurrence and/or mutual exclusivity with the first variant, wherein the quantitative value is based on the covariable information.

11. The method of claim 10, further comprising:
determining a prevalence of at least a second variant in the cfDNA sample, wherein the quantitative value is based further on the prevalence of the second variant.

12. The method of claim 1, wherein the quantitative value is based on the ratio of the first likelihood value to the second likelihood value.

13. The method of claim 1, further comprising determining a level of confidence that the first variant is absent at the clonal level in the cfDNA sample based on the quantitative value.

14. The method of claim 1, further comprising determining a treatment plan to treat a disease in the human subject.

15. The method of claim 14, wherein the disease is cancer.

16. The method of claim 1, further comprising:
determining a prevalence of at least a second variant in the cfDNA sample; and
adjusting the quantitative value based on the prevalence of at least a second variant in the cfDNA sample.

17.-30. (canceled)

31. The method of claim 1, wherein the determination that the first target nucleic acid variant is absent at the first genetic locus in the cfDNA sample indicates that the first genetic locus is wild type.

32. The method of claim 1, wherein the given cancer type is colorectal cancer, wherein the first genetic locus is KRAS, BRAF, or NRAS, and wherein the determination that the first target nucleic acid variant is absent at the first genetic locus in the cfDNA sample indicates that the first genetic locus is wild type KRAS, BRAF, or NRAS.

33. The method of claim 32, further comprising administering Cetuximab and/or Panitumumab to the subject.

34.-97. (canceled)

98. A method of determining that a first variant of interest at a first locus is absent at a clonal level in a cell-free deoxyribonucleic acid (cfDNA) sample of a human subject, the method comprising:
accessing a plurality of sequence reads of the cfDNA sample;
determining that the first variant has not been detected at the first locus in the sample based on the plurality of sequence reads;
generating a first likelihood value based on a probability that the first variant is absent at the clonal level or a second likelihood value based on a probability that the first variant is not absent at the clonal level;
determining a quantitative value based on the first likelihood value or the second likelihood value;
comparing the quantitative value, the first likelihood value, or the second likelihood value to a threshold; and
determining that the first variant of interest at the first locus is absent at the clonal level based on the comparison.
